(19) United States
(12) Patent Application Publication
(10) Pub. No.: US 2011/0139170 A1
(43) Pub. Date: Jun. 16, 2011

(54) NON-SULFATE MILD SURFACTANT SYSTEM FOR SKIN AND HAIR CLEANING

(76) Inventors: Thomas Hippe, Appen (DE); Petra Kursawe, Hamburg (DE)

(21) Appl. No.: 13/030,526

(22) Filed: Feb. 18, 2011

Related U.S. Application Data
(63) Continuation of application No. PCT/EP2009/059495, filed on Jul. 23, 2009.

(30) Foreign Application Priority Data
Aug. 18, 2008 (DE) 10 2008 038 137.3

(51) Int. Cl. A61Q 5/02; A61K 8/60; A61Q 19/10; A61K 8/97; A61K 8/92; A61K 8/64

(52) U.S. Cl. 132/202; 510/126; 510/130; 510/123

(57) ABSTRACT

Non-sulfate skin and hair cleansers that impart advantageous properties to the skin or hair and produce a finely porous and creamy foam, containing the following in a cosmetically acceptable substrate—on a weight basis—(a) at least one acylamino acid salt, (a) at least one alkyl polyglucoside and (b) at least one zwitterionic and/or amphoteric surfactant, wherein the ratio of components (a):(b):(c) is in the range of (0.1-1):(1-3):(0.1-5).

### CROSS REFERENCE TO RELATED APPLICATIONS

The present application is a continuation of International Patent Application No. PCT/EP2009/059495 filed 23 Jul. 2009, which claims priority to German Patent Application No. 10 2008 038 137.3 filed 18 Aug. 2008, both of which are incorporated herein by reference.

The present invention relates to sulfate-free surfactant-containing cleaning agents for skin and/or hair based on a specific, mild surfactant mixture in a defined ratio, use of this surfactant mixture for improving skin compatibility and for manufacturing a micro-porous creamy foam, as well as a method for treating hair and/or skin by use of the agent.

People's constantly increasing hygienic sense and the even greater and broader requirements for cosmetic cleaning agents have continually resulted in new problems and difficulties in their manufacture.

In order to meet consumers' needs, cleaning agents, especially cosmetic cleaning agents for skin and hair, commercially available, for example, as liquid soaps, shampoos, shower baths, foam baths, shower and washing gels, not only have to have a good cleaning power, but should also be well tolerated and not lead to severe degreasing or dryness of the skin or hair even upon frequent use.

Customary cosmetic cleaning products mostly contain anionic surfactants in amounts of about 5 to 20% by weight in order to achieve a satisfactory cleaning performance. Following application of such a cleaning product, however, the consumer often perceives his or her skin and hair condition as not ideal, which is attributed predominantly to the slightly irritating effect of the mostly anionic surfactants. The condition of the skin following application is perceived as dry, taut and sometimes rough, for which subsequent application of creams or lotions is required. The hair feels both dry and brittle and thereby susceptible to hair breakage and split ends. Combability of the hair is also frequently unsatisfactory; therefore, the hair also has to be treated with rinses, cures and tonics after washing.

The consumer, however, does not always wish to carry out this after-treatment step on the skin and/or hair as it involves considerable time and expense.

Thus, many tests have recently been carried out in order to provide agents that not only ensure adequate cleaning, but also provide noticeable conditioning properties to the skin or hair.

Accordingly, various fats, silicones and/or cationic polymers, cationic surfactants, specific plant extracts, etc., have been stably incorporated in cleaning compositions, thereby making available “2 in 1” products that fulfill the above requirements.

A disadvantage of these compositions, however, is that they are still based on anionic (even if mild) sulfate surfactants. The reason for this is that the consumer expects shampoos and/or cleaning compositions to form creamy, micro-porous foam that has a pleasant feel and can be easily dispersed on the skin and/or hair.

Up to now, foam requirements could only be achieved with anionic sulfate surfactants, as the incorporation of care substances into the cosmetic cleaners, particularly fats, often produced a negative effect on foam quality, thereby often requiring an even higher content of sulfate surfactants and additional co-surfactants and foam boosters.

In addition to “2 in 1” products that, if possible, are sulfate-free and form a creamy, micro-porous foam, the manufacture of cosmetic cleaning agents has also shifted more and more towards natural cosmetic products having a high content of active substances of natural (vegetal) origin that basically have not undergone (further) chemical treatment and which are substantially biodegradable and ecologically harmless. The increasing environmental and health awareness of the consumer has contributed decisively to this.

Accordingly, the present invention provides a cosmetic cleaning agent for the skin and hair which meets the above cited requirements.

Ideally, both sulfates and PEG compounds should be dispensed with in the compositions, as PEG compounds often involve substances of natural origin that have been chemically treated. Moreover, some PEG compounds, as emulsifiers for oil and water, tend to make the skin more permeable to harmful substances, which is why they are poorly evaluated in ecological assessments of cosmetic agents.

In addition to an effective cleaning action, the products should strengthen the inner structure of keratinic fibers, thereby improving the suppleness, feel, strength and combability of keratinic fibers.

Completely surprisingly, a novel surfactant mixture has now been found that to a high degree meets the above cited requirements.

This has enabled not only the manufacture of sulfate-free and PEG-free cosmetic agents, but also those that are very mild and which form a particularly creamy, micro-porous and soft foam.

Furthermore, care substances can be stably incorporated into the formulations which afford a sustainable conditioning of the skin and/or of the hair and make a subsequent treatment step with creams, lotions, hair conditioners or rinses unnecessary.

Accordingly, the subject matter of the present invention is sulfate-free cleaning agents for skin and/or hair comprising in a cosmetically acceptable carrier a mixture of mild surfactants containing

(a) at least one acylamino acid salt,
(b) at least one alkyl polyglucoside, and
(c) at least one zwitterionic and/or amphoteric surfactant,
wherein the ratio of components (a):(b):(c) is in the range of (0.1-1):(1-3):(0.1-5).

In a preferred embodiment of the invention, the surfactant ratio (a):(b):(c) is in the range (0.1-1):(1-2):(0.5-3) and especially 1:(1-2):(0.5-2.5).

Besides sulfate-free cosmetic cleaning agents for skin and/or hair, those agents are also inventively particularly preferred that also do not contain PEG compounds.

According to the invention, “PEG-free compounds” are understood to include the following compositions:

(1) compounds of Formula (I)

H—(O—CH₂CH₂)ₙ—OH (I),

wherein n is a whole number from 1 to 100,000,
(2) compounds of Formula (II)

H—(O—CH(CH₃)CH₂)ₙ—OH (II),

wherein n is a whole number from 1 to 100,000,
(3) 3) compounds of Formula (III)

R—(O—CH₂CH₂)ₙ—OH (III),

wherein R is an alkyl or alkenyl group and n is a number from 1 to 10,000,
(4) compounds of Formula (IV)

R—(O—CH₂CH₂)ₙ—OSO₃H (IV),

wherein R is an alkyl or alkenyl group and n is a number from 1 to 10,000, and
(5) compounds of Formula (V)

—(O—CH₂CH₂)ₙ— (V),

wherein n is a whole number from 2 to 100,000.

Agents according to the invention are free of polyethylene glycols according to general Formula (I) (i.e., they comprise neither ethylene glycol (n=1) nor products with a polymerization degree Pₙ=2-4 (diethylene glycol, triethylene glycol and tetraethylene glycol) nor polyethylene glycols with higher degrees Pₙ of ca. 5 to 100,000 which can no longer be manufactured homogeneously, but are rather polydisperse).

They are also preferably free of polypropylene glycols of general Formula (II) (i.e., they comprise neither propylene glycol (n=1) nor products with a polymerization degree Pₙ=2-4 (dipropylene glycol, tripropylene glycol and tetrapropylene glycol) nor polypropylene glycols with higher degrees Pₙ of ca. 5 to 100,000 which can no longer be manufactured homogeneously, but are rather polydisperse).

Addition of ethoxylated compounds should also be avoided. In particular, non-ionic surfactants of the alkyl- or alkenyl ethoxylated type likewise diminish the positive effects of the agent and should therefore not be incorporated into the formulations. Consequently, preferred cleaning agents for skin and/or hair do not contain any compounds of Formula (III), either. As the agents are formulated sulfate-free, they of course do not contain any alkyl ether sulfates of Formula (IV), either.

Preferably, the addition of compounds having ethoxylated groups is totally avoided in agents according to the invention.

Therefore, particularly preferred cleaning agents for skin and/or hair do not contain any compound having the group —(O—CH₂CH₂)ₙ—O— with n=1 to 10,000. Particularly preferred cleaning agents for skin and/or hair do not contain any compounds of Formula (V). The surfactant mixture according to the invention is incorporated into the cleaning agents for skin and/or hair in the abovementioned ratio. Here, individual surfactant components amount to
(a) 0.001 to 15 wt. %, preferably 0.005 to 10 wt. %, more preferably 0.01 to 7.5 wt. % and especially 0.05 to 5 wt. %,
(b) 0.05 to 15 wt. %, preferably 0.1 to 12.5 wt. %, more preferably 0.5 to 10 wt. % and especially 1 to 7.5 wt. %,
(c) 0.05 to 20 wt. %, preferably 0.1 to 15 wt. %, more preferably 0.5 to 12.5 wt. % and especially 1 to 10 wt. %;
   wherein the quantitative data is based on total weight of the cleaning agent for skin and/or hair.

The anionic surfactant component (a) of the surfactant mixture according to the invention contains an acylamino acid salt of Formula (VI),

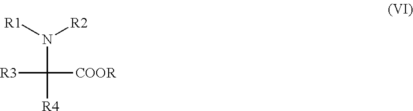

wherein
R is a hydrogen atom, an alkali metal ion or alkaline earth metal ion, an ammonium-, alkylammonium- or alkanolammonium ion,
one of R1 or R2 is hydrogen, a C₁-C₄ alkyl, phenyl or a —CH₂—COOR group, and the other R1 or R2 is a COR′ group, wherein R′ is a straight chain or branched, saturated or unsaturated alkyl group containing 8 to 30 carbon atoms,
R3 and R4 are, independently of one another, hydrogen or linear or branched groups —CH(R5)-(CH₂)ₙ—COOR, wherein R5 is hydrogen or a C₁-C₄ alkyl group and n is a whole number from 0 to 10.

Acylamino acid salts of the amino acids sarcosine, aspartic acid and glutamic acid are preferred and among these, the alkali metal salts of lauroyl sarcosinate, cocoyl sarcosinate, myristoyl sarcosinate, oleyl sarcosinate, lauroyl glutamate, cocoyl glutamate, myristoyl glutamate and stearoyl glutamate are particularly preferred, particularly products marketed under the trade names Protelan® MST 35, Perlastan® C 30, Hamposyl® O, Hamposyl® L 30, Hamposyl® L 95, Eumulgin® SG, Aminsoft® CS 11, Plantapon® ACG 35, Plantapon® ACG 50 and Protelan® AGL 95, wherein for some embodiments of the invention, it can be particularly preferred when the anionic surfactant known under the INCI name Disodium Cocoyl Glutamate is used as component (a). The second essential surfactant of the surfactant mixture according to the invention is an alkyl polyglucoside.

Suitable alkyl polyglycosides (APGs) correspond to the general Formula RO—(Z)ₓ wherein R is a an alkyl, Z is sugar and x is the number of sugar units. APGs according to the invention may simply comprise a defined alkyl group R. However, these compounds are normally manufactured from natural fats and oils or mineral oils. Here, mixtures corresponding to the starting compounds are found as alkyl groups R. Those APGs are particularly preferred, wherein R is essentially C₈ and C₁₀ alkyl groups, essentially C₁₂ and C₁₄ alkyl groups, essentially C₈ to C₁₆ alkyl groups, or essentially C₁₂ to C₁₆ alkyl groups.

Any mono or oligosaccharide can be used as the sugar building block Z. Usually, sugars with 5 or 6 carbon atoms as well as the corresponding oligosaccharides are used. Such sugars include glucose, fructose, galactose, arabinose, ribose, xylose, lyxose, allose, altrose, mannose, gulose, idose, talose and sucrose. Preferred sugar building blocks are glucose, fructose, galactose, arabinose and sucrose, with glucose particularly preferred.

Alkyl polyglycosides used according to the invention contain on average 1.1 to 5 sugar units. Alkyl polyglycosides with x-values of 1.1 to 2.0 are preferred. Alkyl glycosides with x-values of 1.1 to 1.8 are quite particularly preferred.

Particularly preferred alkyl polyglucosides are the products known under the INCI name Lauryl Glucoside, Coco-Glucoside, Decyl Glucoside and C₈-C₁₀ Alkylpolyglucoside, which can also be incorporated into compositions according to the invention as an aqueous solution or as an aqueous solution in mixture with other surfactants. An amphoteric and/or zwitterionic surfactant is added as the third essential surfactant component of the surfactant mixture according to the invention, and is chosen from
N-alkylglycines,
N-alkylpropionic acids,
N-alkylamino butyric acids,
N-alkylimino dipropionic acids,
N-hydroxyethyl-N-alkylamido propylglycines,
N-alkyltaurines,
N-alkylsarcosines,
2-alkylamino propionic acids each with about 8 to 24 carbon atoms in the alkyl group,
2-alkylamino acetic acids each with about 8 to 24 carbon atoms in the alkyl group,
N-cocoalkylamino propionates,
cocoacylaminoethylamino propionates
C₁₂-C₁₈ acylsarcosines,
N-alkyl-N,N-dimethylammonium glycinates, for example, cocoalkyldimethylammonium glycinate,
N-acyl-aminopropyl-N,N-dimethylammonium glycinates, for example cocoacylaminopropyldimethylammonium glycinate,
2-alkyl-3-carboxymethyl-3-hydroxyethyl-imidazolines, each with 8 to 18 carbon atoms in the alkyl or acyl group,
cocoacylaminoethylhydroxyethylcarboxymethyl glycinate
compounds known under the INCI name Cocamidopropyl Betaine,
compounds known under the INCI name Disodium Cocoamphodiacetate, and
compounds known under the INCI name Betaine.

A compound of Formula (IX) is another particularly preferred zwitterionic and/or amphoteric surfactant

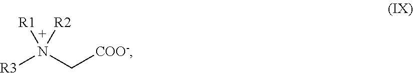

wherein R1 to R3 independently of one another are C₁-C₄ alkyl groups or hydroxyalkyl groups. R1 to R3 are preferably the same groups of methyl or ethyl, and particularly preferably all three groups R1, R2 and R3 are methyl groups. A particularly preferred surfactant (c) is the product of Formula (X), commercialized under the INCI name “Betaine”, for example, the surfactant commercialized under the trade name Tego Natural Betaine® by Goldschmidt (Evonik-Degussa). This surfactant is particularly useful, because it is predominantly of natural origin and, in addition to its surfactant properties, has excellent moisturizing and skin care properties.

In a preferred embodiment of the invention, the cleaning agents for skin and/or hair contain, in addition to the mild surfactant mixture, at least one skin and hair conditioning component corresponding to the following Formula (X):

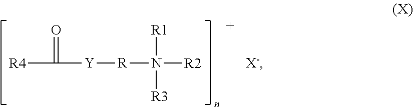

in which
R4 is a long aliphatic hydrocarbon group containing 8 to 24 carbon atoms,
Y is a hetero atom, chosen from O, N or S, wherein, when Y═N, the nitrogen atom carries a further substituent —H or —C₁-C₄ alkyl,
R is a straight chain or branched alkylene group containing 1 to 10 carbon atoms,
R1, R2 and R3 are, independently of one another, C₁-C₈ alkyl, C₁-C₈ hydroxyalkyl or benzyl groups, and
X is an anion such as chloride, bromide, methosulfate, ethosulfate, tosylate, acetate, lactate, phosphate or nitrate.

Those compounds of Formula (X) are preferred wherein
R1, R2 and R3 are identical groups,
X is chloride, bromide or methosulfate,
R is a straight chain alkylene group —(CH₂)ₙ—, wherein n=1, 2 or 3,
Y is —N(R)—, particularly —N(H)— or —N(CH₃), and
R4 is a mixture of different chain lengths from 8 to 24 carbon atoms, as result when the compounds of Formula (IV) are manufactured from naturally occurring raw materials, chosen from tallow oil, coco oil, palm oil and/or soya.

Palmitamidopropyltrimonium chloride, beheneamidopropyltrimonium chloride, palmitamidopropyltrimonium bromide, cocoamidopropyltrimonium chloride, stearylamidopropyltrimonium methosulfate, tallowamidopropyltrimonium chloride and soyamidopropyltrimonium chloride are particularly preferred.

In quite particularly preferred embodiments, the product of Formula (X), commercialized, for example, under the trade name “Varisoft® PATC”, is used and, particularly in the hair cleaning agent, provides a significant improvement in feel and combability of the hair, as well as increasing suppleness. Compounds of Formula (X) are incorporated into the cleaning agents for skin and/or hair in an amount of 0.005 to 10 wt. %, preferably 0.01 to 7.5 wt. %, more preferably 0.05 to 5 wt. % and especially 0.1 to 3 wt. %, based on total weight of the agent.

In another preferred embodiment of the invention, the cleaning agents for skin and/or hair are essentially free of further surfactants. That means that they contain the surfactant mixture only from components (a), (b) and (c). According to the invention, “essentially free” is refers to a maximum content of 4 wt. %, preferably 3 wt. %, more preferably 2.5 wt. % and especially 2 wt. %, wherein the maximum content of additional surfactants is based on total weight of the cleaning agent for skin and/or hair.

Those surfactants incorporated in minor quantities into the cleaning agents for skin and/or hair are mostly ingredients of a commercial product. An additional, optional but preferred component according to the invention is a consistency regulator that is incorporated into the agents in an amount of 0.01 to 10 wt. %, preferably 0.05 to 7.5 wt. %, more preferably 0.1 to 5 wt. % and especially 0.5 to 3 wt. %, based on total weight of the agent. They involve water-soluble and/or partially swellable natural or synthetic polymers that form gels or viscous solutions in aqueous systems. Suitable examples are organic, totally synthetic compounds, such as polyacrylic and polymethacrylic compounds, vinyl polymers, polycarboxylic acids, polyethers, polyimines, polyamides, polyurethanes, inorganic compounds, such as polysilicic acids, clay minerals such as montmorillonites, zeolites, silicas, organic, modified natural products, such as carboxymethyl cellulose and other cellulose ethers, hydroxyethyl- and hydroxypropyl cellulose and microcrystalline cellulose, as well as organic, natural compounds, such as for example agar-agar, carrageen, traganthe, gum arabicum, alginates, pectins, laponites, hectorites, bentonites, polyoses, guar-meal, locust tree bean flour, starches, dextrins, gelatines and casein. In a particularly preferred embodiment, xanthan (E 415), an anionic heteropolysaccharide typically formed by fermenting corn sugar and is isolated as the potassium salt, is used as the consistence regulator. It is produced from Xanthomonas campestris and some other species under aerobic conditions. Xanthan is formed from a chain of linked β-1,4-glucose (cellulose) with side chains. The structure of the sub-groups consists of glucose, mannose, glucuronic acid, acetate and pyruvate. The viscosity of xanthan is determined by the number of pyruvate units. Kelzan ASX-T (available from CP Kelco), for example, is particularly suitable. For further improving the state of the skin and hair, the cleaning agents for skin and/or hair additionally contain preferably at least one further conditioner for skin and/or hair chosen from vegetal oil components, plant extracts and/or protein hydrolyzates.

According to the invention, protein hydrolyzates refer to protein hydrolyzates and/or amino acids and their derivatives (H). Protein hydrolyzates are product mixtures obtained by acid-, base- or enzyme-catalyzed degradation of proteins (albumins). Molecular weight of usable protein hydrolyzates ranges from 75, the molecular weight of glycine, to 200,000, preferably the molecular weight is 75 to 50,000 and quite particularly preferably 75 to 20,000 Dalton.

According to the invention, added protein hydrolyzates can be of vegetal as well as of animal or marine or synthetic origin.

Animal protein hydrolyzates include elastin, collagen, keratin, silk protein, and milk albumin protein hydrolyzates, which can also be present in the form of their salts. Such products are marketed, for example, under the trade names Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex) and Kerasol® (Croda).

According to the invention, it is preferred to use protein hydrolyzates of vegetal origin, for example, protein hydrolyzates of soya, almond, pea, potato and wheat. Such products are available, for example, under the trade names Gluadin® (Cognis), DiaMin® (Diamalt), Lexein® (Inolex) Hydrosoy® (Croda), Hydrolupin® (Croda), Hydrosesame® (Croda), Hydrotritium® (Croda) and Crotein® (Croda).

Preparations according to the invention preferably contain protein hydrolyzates or their derivatives in amounts of 0.1 to 10% by weight, based on total agent. Quantities of 0.05 to 7.5, particularly 0.1 to 5% by weight, are preferred. Normally, triglycerides and mixtures of triglycerides are used as the natural (vegetal) oils. Preferred natural oils include coconut oil, (sweet) almond oil, walnut oil, pear kernel oil, apricot kernel oil, avocado oil, tea tree oil, soybean oil, sesame oil, sunflower oil, camellia oil, evening primrose oil, rice grain oil, palm kernel oil, mango kernel oil, cuckoo flower oil, almond oil, macadamia nut oil, grape seed oil, amaranth seed oil, argan oil, babssu oil, olive oil, wheat germ oil, pumpkin seed oil, abutilon seed oil, hazelnut oil, safflower oil, canola oil, sasanqua oil, jojoba oil, cocoa butter and shea butter. Vegetal oils are preferably incorporated in the agents in amounts of 0.1 to 10 wt. %, based on total agent. Quantities of 0.05 to 7.5, particularly 0.1 to 5% by weight, are preferred.

Preferred agents according to the invention contain plant extracts of green tea, oak bark, stinging nettle, hamamelis, hops, henna, chamomile, burdock root, field horsetail, hawthorn, linden blossom, almonds, aloe vera, spruce needles, horse chestnut, sandal wood, juniper, coconut, pomegranate, mango, apricot, lime, wheat, kiwi, melon, orange, grapefruit, elder, sage, rosemary, birch, mallow, lady's smock, rooibos, quendel, common yarrow, thyme, lemon balm, rest-harrow, coltsfoot, vanilla, marshmallow (althea), meristem, ginseng and/or ginger.

Usually, these extracts are manufactured by extraction of the whole plant. In certain cases, however, it can also be preferred to produce the extracts solely from blossoms and/or leaves of the plant.

The extraction composition used to prepare the cited plant extracts can be water, alcohols, or mixtures of water and alcohol. Preferred alcohols include lower alcohols such as ethanol and isopropanol, particularly polyhydric alcohols such as ethylene glycol, propylene glycol and butylene glycol, both as the sole extraction agent as well as in aqueous mixtures. Plant extracts based on water/propylene glycol in the ratio 1:10 to 10:1 are particularly suitable.

According to the invention, plant extracts can be used in pure as well as diluted form. When they are used in diluted form, they normally contain ca. 2-80% by weight active substance, and the solvent is the extraction agent or mixture of extraction agents used for their extraction.

Plant extracts are added to the agents in an amount of 0.01 to 5% by weight, preferably 0.02 to 4% by weight and particularly 0.05 to 3% by weight, based on total weight of the agent.

The conditioning effects of the agents can be enhanced even further by adding conditioning enhancers. Preferably, these are chosen from certain groups of known conditioners, because these conditioning enhancers harmonize exceptionally well with the inventive surfactant mixture, both with respect to formulation technology and conditioning effect. Preferred cleaning agents for skin and/or hair additionally contain at least one conditioning enhancer from the group L-carnitine and/or its salts; panthenol and/or pantothenic acid; 2-furanone and/or its derivatives, especially pantolactone; taurine and/or its salts; niacinamide; ubiquinone; ectoin; and allantoin.

In agents of this embodiment, the surfactant mixture is combined with at least one conditioning enhancer chosen from L-carnitine and/or its salts; panthenol and/or pantothenic acid; 2-furanones and/or its derivatives, especially pantolactone, taurine and/or its salts; niacinamide; ubiquinones; ectoin; allantoin, extracts of Echinacea. These conditioning enhancers are described below. L-Carnitine (IUPAC name (R)-(3-carboxy-2-hydroxypropyl)-N,N,N-trimethylammonium hydroxide), is a naturally occurring, vitamin-like substance.

As a betaine, L-carnitine can form addition compounds and double salts. Preferred L-carnitine derivatives are chosen from acetyl-L-carnitine, L-carnitine fumarate, L-carnitine citrate, lauroyl L-carnitine, and particularly preferably L-carnitine tartrate. The L-carnitine compounds can be obtained, for example, from Lonza GmbH. Preferred cleaning agents for skin and/or hair contain 0.001 to 10 wt. %, preferably 0.005 to 7.5 wt. %, particularly preferably 0.01 to 5 wt. % and particularly 0.05 to 2.5 wt. % L-carnitine or L-carnitine derivatives, based on total weight of the agent, wherein preferred L-carnitine derivatives are chosen from acetyl-L-carnitine, L-carnitine fumarate, L-carnitine citrate, lauroyl L-carnitine and in particular L-carnitine tartrate.

Panthenol (IUPAC name: (+)-(R)-2,4-dihydroxy-N-(3-hydroxypropyl)-3,3-dimethylbutyramide) is converted to pantothenic acid in the body. Pantothenic acid is a vitamin from the vitamin B group (vitamin B5).

Inventively preferred cleaning agents for skin and/or hair contain, based on total weight of the agent, 0.01 to 5 wt. %, preferably 0.05 to 2.5 wt. %, particularly preferably 0.1 to 1.5 wt. % and particularly 0.25 to 1 wt. % panthenol ((±)-2,4-dihydroxy-N-(3-hydroxypropyl)-3,3-dimethylbutyramide). Preferred cleaning agents for skin and/or hair contain, based on total weight of the agent, 0.01 to 15 wt. %, preferably 0.025 to 12.5 wt. %, particularly preferably 0.05 to 10 wt. %, more preferably 0.1 to 7.5 wt. % and particularly 0.5 to 5 wt. % of at least one 2-furanone derivative of the Formula (Fur-I) and/or of the Formula (Fur-II)

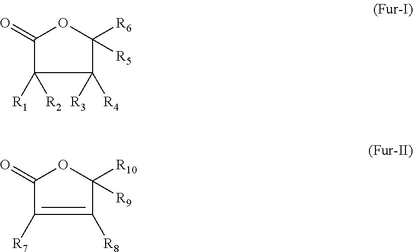

wherein R¹ to R¹⁰, independently of one another, are:
hydrogen, —OH, a methyl, methoxy, aminomethyl or hydroxymethyl group,
—C₂-C₄ saturated or mono- or di-unsaturated, branched or linear hydrocarbon groups,
—C₂-C₄ saturated or mono- or di-unsaturated, branched or linear mono-, di- or trihydroxyhydrocarbon groups,
—C₂-C₄ saturated or mono or di-unsaturated, branched or linear mono-, di- or triamino hydrocarbon groups,
an —OR¹¹ group, with —R¹¹ as a —C₂-C₄ saturated or mono or di-unsaturated, branched or linear hydrocarbon group, —C₂-C₄ saturated or mono- or di-unsaturated, branched or linear mono-, di- or trihydroxyhydrocarbon groups,
an —NR¹²R¹³ group, wherein R¹² and R¹³ are, independently of one another, hydrogen, a methyl, a —C₂-C₄ saturated or mono- or di-unsaturated, branched or linear hydrocarbon group, a —C₂-C₄ saturated or mono- or di-unsaturated, branched or linear mono-, di- or trihydroxyhydrocarbon group,
a —COOR¹⁴ group, wherein R¹⁴ is hydrogen, a methyl, a —C₂-C₄ saturated or mono- or di-unsaturated, branched or linear hydrocarbon group, a —C₂-C₄ saturated or mono- or di-unsaturated, branched or linear mono-, di- or trihydroxyhydrocarbon group, a C₂-C₄ saturated or mono- or di-unsaturated, branched or linear mono-, di- or triamino hydrocarbon group,
a —CONR¹⁵R¹⁶ group, wherein R¹⁵ and R¹⁶ are hydrogen, methyl, a —C₂-C₄ saturated or mono- or di-unsaturated, branched or linear hydrocarbon group, a —C₂-C₄ saturated or mono- or di-unsaturated, branched or linear mono-, di- or trihydroxyhydrocarbon group, a —C₂-C₄ saturated or mono- or di-unsaturated, branched or linear mono-, di- or triamino hydrocarbon group,
a —COR¹⁶ group, wherein R¹⁶ is hydrogen, a methyl, a —C₂-C₄ saturated or mono- or di-unsaturated, branched or linear hydrocarbon group, a —C₂-C₄ saturated or mono- or di-unsaturated, branched or linear mono- di- or trihydroxyhydrocarbon group, a —C₂-C₄ saturated or mono- or di-unsaturated, branched or linear mono-, di- or triamino hydrocarbon group,
an —OCOR¹⁷ group, wherein R¹⁷ is a methyl, a —C₂-C₃₀ saturated or mono- or polyunsaturated, branched or linear hydrocarbon group, a —C₂-C₃₀ saturated or mono- or polyunsaturated, branched or linear mono- di-, tri- or polyhydroxyhydrocarbon group, a —C₂-C₃₀ saturated or mono- or poly-unsaturated, branched or linear mono-, di-, tri- or polyamino hydrocarbon group,
   with the proviso that when R⁷ and R⁸ are —OH and at the same time R⁹ or R¹⁰ os hydrogen, then the remaining group R⁹ or R¹⁰ is not a dihydroxyethyl group. In a particularly preferred embodiment, a compound of formula (Fur-I) is employed. Preferably, in a compound of formula (Fur-I), R¹ and R², independently of one another, are:
hydrogen, an —OH, a methyl, methoxy, aminomethyl, hydroxymethyl group,
a —C₂-C₄ saturated or mono- or di-unsaturated, branched or linear mono-, di- or trihydroxyhydrocarbon group,
an —OR¹¹ group, with —R¹¹ as a —C₂-C₄ saturated or mono- or di-unsaturated, branched or linear hydrocarbon group, —C₂-C₄ saturated or mono- or di-unsaturated, branched or linear mono-, di- or trihydroxyhydrocarbon group,
a —COOR¹⁴ group, wherein R¹⁴ is hydrogen, a methyl, a —C₂-C₄ saturated or mono- or di-unsaturated, branched or linear hydrocarbon group, a —C₂-C₄ saturated or mono- or di-unsaturated, branched or linear mono- di- or trihydroxyhydrocarbon group,
a —COOR¹⁶ group, wherein R¹⁶ is a methyl, a —C₂-C₄ saturated or mono- or di-unsaturated, branched or linear hydrocarbon group, a —C₂-C₄ saturated or mono- or di-unsaturated, branched or linear mono- di- or trihydroxyhydrocarbon group,
an —OCOR¹⁷ group, wherein R¹⁷ is methyl, a —C₂-C₃₀ saturated or mono- or polyunsaturated, branched or linear hydrocarbon group, a C₂-C₃₀ saturated or mono- or polyunsaturated, branched or linear mono-, di-, tri- or polyhydroxyhydrocarbon group.

In a particularly preferred embodiment, the added compound corresponding to formula (Fur-I) is employed
(R)-(−)-4-hydroxymethyl-γ-butyrolactone and/or
D,L-4-hydroxymethyl-γ-butyrolactone and/or
(S)-(+)-4-hydroxymethyl-γ-butyrolactone and/or
R-(−)-2-hydroxy-3,3-dimethyl-γ-butyrolactone and/or
D,L-2-hydroxy-3,3-dimethyl-γ-butyrolactone and/or
S(+)-2-hydroxy-3,3-dimethyl-γ-butyrolactone and/or
4-hydroxy-2,5-dimethyl-3(2H)-furanone and/or
tetrahydro-5-oxo-2-furancarboxylic acid and/or
tetrahydro-5-oxo-2-furancarboxylic acid, Na salt and/or
tetrahydro-5-oxo-2-furancarboxylic acid, K salt and/or
2,5-dihydro-5-methoxy-2-furanone and/or
dihydro-3-hydroxy-4,4-dimethyl-2(3H)-furanone.

In a quite particularly preferred embodiment, dihydro-3-hydroxy-4,4-dimethyl-2(3H)-furanone is used as the compound corresponding to Formula (Fur-I).

A further preferred employable conditioning enhancer having activating properties is taurine. Preferred cleaning agents for skin and/or hair contain, based on total weight of the agent, 0.01 to 15 wt. %, preferably 0.025 to 12.5 wt. %, more preferably 0.05 to 10 wt. %, even more preferably 0.1 to 7.5 wt. %, and particularly 0.5 to 5 wt. % of taurine (2-aminoethane sulfonic acid).

Vitamins, provitamins or vitamin precursors are a further preferred group of conditioning enhancers in the agents. They are described below: Retinol (vitamin A₁) as well as 3,4-Didehydroretinol, (vitamin A₂) belong in the group of substances designated as vitamin A. β-carotene is the provitamin of retinol. Examples of suitable vitamin A components according to the invention are vitamin A acid and its esters, vitamin A aldehyde and vitamin A alcohol as well as its esters such as the palmitate and acetate. The agents preferably contain vitamin A components in amounts of 0.05 to 1% by weight, based on total preparation.

The vitamin B group or the vitamin B complex include inter alia
vitamin B₁ (thiamin)
vitamin B₂ (riboflavin)
Vitamin B₃. The compounds nicotinic acid and nicotinamide (niacinamide) are often included under this designation. According to the invention, nicotinamide is preferred and is comprised in the compositions used according to the invention in amounts of 0.05 to 1 wt. % based on the total composition.
Vitamin B₆ (pyridoxine as well as pyridoxamine and pyridoxal).

Vitamin C (ascorbic acid). Vitamin C is preferably added to compositions according to the invention in amounts of 0.1 to 3 wt. %, based on total composition. Its use in the form of the palmitate ester, glucosides or phosphates can be preferred. The use in combination with tocopherols can also be preferred. Vitamin E (Tocopherols, especially α-tocopherol). Tocopherol and its derivatives, which include in particular esters such as acetate, nicotinate, phosphate and succinate, are used in the compositions preferably in amounts of 0.05-1 wt. %, based on total composition.

Vitamin F. The term “vitamin F” typically refers to essential fatty acids, particularly linoleic acid, linolenic acid and arachidonic acid.

Vitamin H. The compound (3aS,4S,6aR)-2-oxohexahydrothienol[3,4-d]imidazole-4-valeric acid represents Vitamin H, for which the trivial name biotin has become accepted. The compositions preferably contain biotin in amounts of 0.0001 to 1.0 wt. %, particularly in amounts of 0.001 to 0.01 wt. %, based on total weight of the composition.

In summary, preferred cleaning agents for skin and/or hair contain, based on total weight of the agent, 0.1 to 5 wt. %, preferably 0.2 to 4 wt. %, more preferably 0.25 to 3.5 wt. %, particularly preferably 0.5 to 3 wt. % and particularly 0.5 to 2.5 wt. % of vitamins and/or provitamins and/or vitamin precursors, which are preferably attributed to the groups A, B, C, E, F and H, wherein preferred agents contain panthenol ((±)-2,4-dihydroxy-N-(3-hydroxypropyl)-3,3-dimethylbutyramide, provitamin B5) and/or pantothenic acid (Vitamin B₃, Vitamin B₅) and/or niacin, niacinamide or nicotinamide (Vitamin B₃) and/or L-ascorbic acid (Vitamin C) and/or thiamine (Vitamin B₁) and/or riboflavin (Vitamin B₂, Vitamin G) and/or biotin (Vitamin B₇, Vitamin H) and/or folic acid (Vitamin B₉, Vitamin B_{c} or Vitamin M) and/or Vitamin B₆ and/or Vitamin B₁₂.

It has been shown that certain quinones have a particular aptitude as a conditioning enhancer. The agents can therefore contain 0.0001 to 5 wt. % of at least one bioquinone of formula (Ubi) as an additional conditioning enhancer

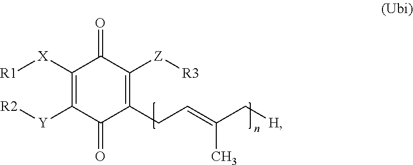

wherein
X, Y, z independently of each other, are —O— or —NH— or NR⁴— or a chemical bond
R¹, R², R³ independently of one another, are a hydrogen atom or an optionally substituted aryl group or an optionally substituted (C₁-C₆) alkyl group or a hydroxyalkyl group or a polyhydroxyalkyl group or an optionally substituted (C₁-C₆) alkylene group, or a (C₁-C₆) acyl group, wherein preferred groups independently of one another are chosen from —H, —CH₃, —CH₂CH₃, —(CH₂)₂CH₂, —CH(CH₃)₂, —(CH₂)₃CH₃, —CH(CH₃)CH₂CH₃, —CH₂CH(CH₃)₂, —C(CH₃)₃
R⁴ is —CH₃, —CH₂CH₃, —(CH₂)₂CH₂, —CH(CH₃)₂, —(CH₂)₃CH₃, —CH(CH₃)CH₂CH₃, —CH₂CH(CH₃)₂, —C(CH₃)₃
n is values from 1 to 20, preferably from 2 to 15 and in particular for 5, 6, 7, 8, 9, 10.

Particularly preferred cleaning agents for skin and/or hair contain, based on total weight of the agent, 0.0001 to 1 wt. %, preferably 0.001 to 0.5 wt. % and particularly preferably 0.005 to 0.1 wt. % of at least one ubiquinone and/or at least one ubiquinol and/or at least one derivative of these substances, wherein preferred agents contain an ubiquinone of Formula (Ubi)

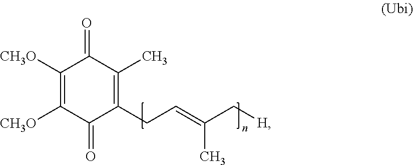

wherein n is the value 6, 7, 8, 9 or 10, preferably 10 (Coenzyme Q10). The agents can contain ectoin ((4S)-2-methyl-1,4,5,6-tetrahydropyrimidine-4-carboxylic acid) as an additional conditioning enhancer. Preferred cleaning agents for hair contain, based on total weight of the agent, 0.001 to 10 wt. %, preferably 0.01 to 5 wt. %, particularly preferably 0.05 to 2.5 wt. % and especially 0.1 to 1 wt. % (S)-2-methyl-1,4,5,6-tetrahydro-4-pyrimidine carboxylic acid (ectoin) as well as the physiologically acceptable salts of this compound and/or (S,S)-5-hydroxy-2-methyl-1,4,5,6-tetrahydro-4-pyrimidine carboxylic acid (hydroxyectoin) as well as the physiologically acceptable salts of this compound.

Another conditioning enhancer is allantoin (5-ureidohydantoin, N-(2,5-dioxo-4-imidazolidinyl)urea) that is used in the cosmetic field in skin creams, sun tan protections, aftershaves, tooth creams and in agents against excessive hyperhidrosis and skin irritations. It accelerates cell construction, cell formation or cell regeneration and calms the skin. Allantoin also supports the healing of difficult-to-heal wounds, although it possesses no antiseptic properties.

Particularly preferred cleaning agents for skin and/or hair contain, based on total weight of the agent, 0.001 to 10 wt. %, preferably 0.01 to 5 wt. %, particularly preferably 0.05 to 2.5 wt. % and particularly 0.1 to 1 wt. % of 5-ureidohydantoin (allantoin). In order to improve the elasticity and consolidation of the internal structure of hair treated with the agents, the agents can contain purine and/or purine derivatives as a conditioning enhancer. In particular, the combination of purine and/or purine derivatives with ubiquinones and/or plastoquinones as the conditioning enhancer means that hair treated with corresponding agents demonstrate inter alia higher measured values in differential thermo analysis and improved wet and dry combability. Inventively preferred cosmetic agents contain, based on total weight of the agent, 0.001 to 2.5 wt. %, preferably 0.0025 to 1 wt. %, particularly preferably 0.005 to 0.5 wt. % and particularly 0.01 to 0.1 wt. % purine(s) and/or purine derivative(s) of the Formula (Pur-I)

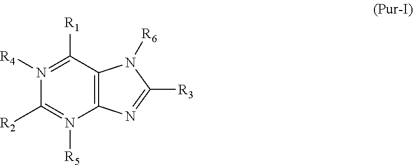

wherein R¹, R² and R³ are, independently of one another, chosen from —H, —OH, NH₂, —SH, and R⁴, R⁵ and R⁶ are, independently of one another, chosen from —H, —CH₃ and —CH₂—CH₃, wherein the following compounds are preferred:
Purine (R¹═R²═R³═R⁴═R⁵═R⁶═H)
Adenine (R¹═NH₂, R²═R³═R⁴═R⁵═R⁶═H)
Guanine (R¹═OH, R²═NH₂, R³═R⁴═R⁵═R⁶═H)
Uric acid (R¹═R²═R³═OH, R⁴═R⁵═R⁶═H)
Hypoxanthine (R¹═OH, R²═R³═R⁴═R⁵═R⁶═H)
6-Purine thiol (R¹═SH, R²═R³═R⁴═R⁵═R⁶═H)
6-Thioguanine (R¹═SH, R²═NH₂, R³═R⁴═R⁵═R⁶═H)
Xanthine (R¹═R²═OH, R³═R⁴═R⁵═R⁶═H)
Caffeine (R¹═R²═OH, R³═H, R⁴═R⁵═R⁶═CH₃)
Theobromine (R¹═R²═OH, R³═R⁴═H, R⁵═R⁶═CH₃)
Theophylline (R¹═R²═OH, R³═H, R⁴═CH₃, R⁵═CH₃, R⁶═H).

It is of further advantage to use purine or purine derivatives and bioquinones in a specific ratio to one another. Here, agents according to the invention are preferred wherein the weight ratio of purine (derivative(s)) and bioquinone(s) is 10:1 to 1:100, preferably 5:1 to 1:50, particularly preferably 1:1 to 1:10. As already mentioned, caffeine is a particularly preferred purine derivative and coenzyme Q10 is a particularly preferred bioquinone. Accordingly, particularly preferred inventive agents comprise, based on their weight, 0.001 to 2.5 wt. %, preferably 0.0025 to 1 wt. %, particularly preferably 0.005 to 0.5 wt. % and especially 0.01 to 0.1 wt. % caffeine and 0.0002 to 4 wt. %, preferably 0.0005 to 3 wt. %, particularly preferably 0.001 to 2 wt. %, more preferably 0.0015 to 1 and especially 0.002 to 0.5 wt. % coenzyme Q10.

The agents can also include flavonoids as the conditioning enhancer. Flavonoids can be used from all six groups, wherein certain representatives from the individual groups are preferred as the conditioning enhancer due to their particularly intensive action. Preferred flavonols are quercetin, rutin, kaempferol, myricetin, isorhamnetin, preferred flavanols are catechin, gallocatechin, epicatechin, epigallocatechin gallate, theaflavin, thearubigin, preferred flavones are luteolin, apigenin, morin, preferred flavanones are hesperetin, naringenin, eriodictyol, preferred isoflavonoids are genistein, daidzein, and preferred anthocyanidines (anthocyans) are cyanidin, delphinidin, malvidin, pelargonidin, peonidin, petunidin.

Accordingly, particularly preferred inventive cleaning agents for skin and/or hair contain, based on total weight of the agent, 0.001 to 2.5 wt. %, preferably 0.0025 to 1 wt. %, particularly preferably 0.005 to 0.5 wt. % and especially 0.01 to 0.1 wt. % flavonoids, especially flavonols, particularly preferably 3,3′,4′,5,7-pentahydroxyflavone (quercetin) and/or 3,3′,4′,5,7-pentahydroxyflavone-3-O-rutinosid (rutin). The additional use of bisabolol and/or bisabolol oxides as the conditioning enhancer in the agents is also preferred. Here, cleaning agents for skin and/or hair are preferred which additionally contain 0.001 to 5 wt. %, preferably 0.01 to 4 wt. %, particularly preferably 0.02 to 2.5 wt. % and especially 0.1 to 1.5 wt. % bisabolol and/or oxides of bisabolol, preferably (−)-alpha-bisabolol. Creatin is also suitable as the conditioning enhancer. Creatin (3-methylguanidino acetic acid) is an organic acid that contributes inter alia to the energy supply of muscles in vertebrates. Creatin is synthesized in the kidneys, the liver and in the pancreas. It is formally derived from the amino acids glycine and arginine and is present up to 95% in the muscle of the skeleton. Particularly preferred cleaning agents for skin and/or hair contain, based on total weight of the agent, 0.01 to 15 wt. %, preferably 0.025 to 12.5 wt. %, particularly preferably 0.05 to 10 wt. %, more preferably 0.1 to 7.5 wt. % and particularly 0.5 to 5 wt. % of N-methylguanidino acetic acid (creatin). The agents (in so far as they are presented as cleaning agents for hair) can contain, in addition to the above cited ingredients and optional further ingredients, additional substances that prevent, mitigate or heal hair loss. A content of hair root-stabilizing active substances is particularly advantageous. These substances will be described below:

Presently, Propecia (Finasterid) is the only preparation approved worldwide and for which an efficacy and tolerance has been proven in numerous studies. Propecia acts so that less DHT is formed from testosterone.

Minoxidil, with or without supplementary additives, is probably the oldest demonstrably effective hair growth agent. It can only be used externally to treat hair loss. There exist hair tonics having 2%-5% Minoxidil and gels with up to 15% Minoxidil. Efficacy increases with the dose, although Minoxidil is only soluble to 5% in hair tonics. Hair tonics with up to 2% Minoxidil content are obtainable without prescription in many countries. Spironolactone in the form of hair tonic and in combination with Minoxidil can be used for external application in order to combat the hormonal effects on the hair follicle. Spironolactone acts as an androgen receptor blocker (i.e., the binding of DHT onto the hair follicle is impeded).

In summary, preferred hair treatment agents according to the invention additionally contain, based on total weight of the agent, 0.001 to 5 wt. % hair root-stabilizing substance, especially Minoxidil and/or Finasterid and/or Ketoconazol.

Additional anti-dandruff active substances specifically reduce the amount of yeast that causes dandruff, bacterial flora returns to normal percentage composition and desquamation is reduced to the physiological level. However, laboratory tests have shown that different varieties of the Pityrosporum ovale react with varying degrees of success to the anti-dandruff active substances. In order to best combat all dandruff pathogens, a combination of anti-dandruff active substances is therefore most successful. In summary, preferred inventive cleaning agents for hair comprise 0.05 to 5 wt. %, preferably 0.1 to 3.0 wt. % and especially 0.3 to 2.0 wt. % (based on total weight of the agent). Preferred anti-dandruff agents are chosen from Piroctone Olamine, Climbazol, Zink Pyrithion, Ketoconazole, salicylic acid, sulfur, selenium sulfides, tar preparations, undecenoic acid derivatives, extracts of burdock root, poplar, stinging nettle, walnut shells, birch, willow bark, rosemary and/or arnica.

Salicylic acid, Climbazol, Zink Pyrithion and Piroctone Olamine are preferred. In addition to the conditioning enhancers, the agents can contain additional conditioning substances. Their presence is not absolutely necessary for achieving the inventive effect; however, additional effects can occur from use of these conditioning substances, such as a pleasant hold or a pleasant application feel.

The agents can particularly advantageously contain one or more amino acids as additional ingredients. Particularly preferred amino acids derive from the group glycine, alanine, valine, leucin, isoleucin, phenylalanine, tyrosine, tryptophan, proline, aspartic acid, glutamic acid, asparagine, glutamine, serine, threonine, cystein, methionine, lysine, arginine, histidine, β-alanine, 4-amino butyric acid (GABA), betaine, L-cystine (L-Cyss), L-carnitine, L-citrulline, L-theanine, 3′,4′-dihydroxy-L-phenylalanine (L-Dopa), 5′-hydroxy-L-tryptophan, L-homocystein, S-methyl-L-methionine, S-allyl-L-cysteine sulfoxide (L-alliin), L-trans-4-hydroxyproline, L-5-oxoproline (L-pyroglutamic acid), L-phosphoserine, creatine, 3-methyl-L-histidin, L-ornithine, wherein both the individual amino acids as well as mixtures can be used.

Preferred agents contain one or more amino acids in narrow quantitative ranges. Here, inventively preferred cleaning agents for skin and/or hair contain, based on total weight of the agent, 0.01 to 5 wt. %, preferably 0.02 to 2.5 wt. %, particularly preferably 0.05 to 1.5 wt. % more preferably 0.075 to 1 wt. % and particularly 0.1 to 0.25 wt. % of amino acid(s) as the care substance, preferably from the group of glycine and/or alanine and/or valine and/or lysine and/or leucine and/or threonine.

The agents can have at least one carbohydrate from the group of monosaccharides, disaccharides and/or oligosaccharides as a further ingredient. Here, inventively preferred cleaning agents for skin and/or hair contain, based on total weight of the agent, 0.01 to 5 wt. %, preferably 0.05 to 4.5 wt. %, particularly preferably 0.1 to 4 wt. % more preferably 0.5 to 3.5 wt. % and particularly 0.75 to 2.5 wt. % of carbohydrate(s) chosen from monosaccharides, disaccharides and/or oligosaccharides, wherein preferred carbohydrates are chosen from
monosaccharides, especially D-ribose and/or D-xylose and/or L-arabinose and/or D-glucose and/or D-mannose and/or D-galactose and/or D-fructose and/or sorbose and/or L-fucose and/or L-rhamnose;
disaccharides, especially saccharose and/or maltose and/or lactose and/or trehalose and/or cellobiose and/or gentiobiose and/or isomaltose.

Silicones represent a particularly preferred group of ingredients. Preferred inventive agents comprise at least one silicone, preferably a silicone chosen from:
(i) volatile or non-volatile, linear or cyclic, crosslinked or non-crosslinked polyalkylsiloxanes, polyarylsiloxanes, polyalkylarylsiloxanes,
(ii) polysiloxanes having one or more organofunctional groups in their general structure, chosen from:
   (a) substituted or unsubstituted aminated groups;
   (b) (per)fluorinated groups;
   (c) thiol groups;
   (d) carboxylate groups;
   (e) hydroxylated groups;
   (f) alkoxylated groups;
   (g) acyloxyalkyl groups;
   (h) amphoteric groups;
   (i) bisulfite groups;
   (j) hydroxyacylamino groups;
   (k) carboxyl groups;
   (l) sulfonic acid groups; and
   (m) sulfate or thiosulfate groups;
(iii) linear polysiloxane(A)-polyoxyalkylene(B) block copolymers of the type (A-B)ₙ with n>3;
(iv) grafted silicone polymers with a non-silicone-containing organic backbone having an organic main chain formed from organic monomers that do not contain silicone, onto which at least one polysiloxane macromer is grafted in the chain as well as optionally on at least one chain end;
(v) grafted silicone polymers containing a polysiloxane backbone, onto which are grafted non-silicone-containing organic monomers, and which have a polysiloxane main chain, onto which at least one organic macromer having no silicone is grafted in the chain as well as optionally on at least one of their chain ends;
   or their mixtures.

Particularly preferred agents contain silicone(s) preferably in amounts of 0.1 to 10 wt. %, preferably 0.25 to 7 wt. % and especially from 0.5 to 5 wt. %, based on total agent.

The silicones are preferably water-soluble. Preferred agents contain at least one water-soluble silicone. “Clear” products are often preferred by consumers on aesthetic grounds. Accordingly, preferred hair treatment agents are therefore transparent or translucent.

According to the present invention, a composition is transparent or translucent when its NTU value is below 100. The NTU-unit (Nephelometry Turbidity Unit; NTU) is a unit used in water treatment for turbidity measurements in liquids. It is the unit of turbidity of a liquid measured with a calibrated nephelometer. Moreover, in a preferred embodiment of the invention, an agent can also contain UV filters (I). Useful UV filters are typically not limited with respect to structure and physical properties. Indeed, all UV filters that can be used in the cosmetic field having an absorption maximum in the UVA (315-400 nm), UVB (280-315 nm) or UVC (<280 nm) regions are suitable. UV filters having an absorption maximum in the UVB region, especially in the range from about 280 to about 300 nm, are particularly preferred.

UV-filters used in accordance with the invention include substituted benzophenones, p-aminobenzoate esters, diphenylacrylate esters, cinnamate esters, salicylate esters, benzimidazoles and o-aminobenzoate esters. Those UV filters with a molecular extinction coefficient at the absorption maximum of 15,000 or greater, particularly 20,000 or greater, are preferred.

According to a further embodiment, those UV filters are preferred which have a cationic group, especially a quaternary ammonium group. Two preferred UV filters containing cationic groups are the commercially available compounds cinnamic acid amidopropyltrimethylammonium chloride (Incroquat® UV-283) and dodecyldimethylaminobenzamidopropyldimethylammonium tosylate (Escalol® HP 610). Compositions according to the invention preferably contain UV filters (I) in quantities of 0.01 to 5 wt. %, based on total composition. Quantities of 0.4-2.5 wt. % are preferred.

The agents can further include penetration aids and/or swelling agents (M). These include urea and urea derivatives, guanidine and its derivatives, arginine and its derivatives, water glass, imidazole and its derivatives, histidine and its derivatives, benzyl alcohol, glycerin, glycol and glycol ethers, propylene glycol and propylene glycol ethers, for example, propylene glycol monoethyl ether, carbonates, hydrogen carbonates, diols and triols, and particularly 1,2-diols and 1,3-diols such as for example 1,2-propane dial, 1,2-pentane diol, 1,2-hexane diol, 1,2-dodecane diol, 1,3-propane diol, 1,6-hexane diol, 1,5-pentane diol, 1,4-butane diol.

In the context of the invention, short chain carboxylic acids (N) can advantageously support the complex of active substances (A). Short chain carboxylic acids and their derivatives refer to carboxylic acids that can be saturated or unsaturated and/or linear or branched or cyclic and/or aromatic and/or heterocyclic and have a molecular weight of less than 750. Saturated or unsaturated or linear or branched carboxylic acids with a chain length of 1 to 16 carbon atoms in the chain are preferred. Those with a chain length of 1 up to 12 carbon atoms in the chain are quite particularly preferred.

In the context of the invention, short chain carboxylic acids can have one, two, three or more carboxyl groups. Carboxylic acids with a plurality of carboxyl groups are preferred, particularly di- and tricarboxylic acids. The carboxyl groups can be totally or partially present as an ester, acid anhydride, lactone, amide, imide acid, lactam, lactim, dicarboximide, carbohydrazide, hydrazone, hydroxam, hydroxime, amidine, amidoxime, nitrile, phosphonate- or phosphate ester. The carboxylic acids can be substituted along the carbon chain or on the cyclic structure. Substituents of the carboxylic acids include C1-C8 alkyl, C2-C8 alkenyl, aryl, aralkyl and aralkenyl, hydroxymethyl, C2-C8 hydroxyalkyl, C2-C8 hydroxyallcenyl, aminomethyl, C2-C8 aminoalkyl, cyano, formyl, oxo, thioxo, hydroxyl, mercapto, amino, carboxyl or imino groups. Preferred substituents are C1-C8 alkyl, hydroxymethyl, hydroxyl, amino and carboxyl groups. Substituents in the α-position are particularly preferred. Quite particularly preferred substituents are hydroxyl, alkoxy and amino groups, wherein the amino function can be optionally further substituted by alkyl, aryl, aralkyl and/or alkenyl groups. In addition, equally preferred carboxylic acid derivatives are the phosphonate- and phosphate esters.

In a further preferred embodiment, the inventive agents can comprise emulsifiers (F). Emulsifiers act at the interface to produce water or oil-stable adsorption layers that protect the dispersed droplets against coalescence and thereby stabilize the emulsion. Thus, emulsifiers, like surfactants, are composed of hydrophobic and hydrophilic molecular moieties. Hydrophilic emulsifiers preferably form O/W emulsions and hydrophobic emulsifiers preferably form W/O emulsions. An emulsion is understood to mean a dispersion of a liquid in the form of droplets in another liquid using an energy input to afford interfaces stabilized with surfactants. The choice of this emulsifying surfactant or emulsifier depends on the materials being dispersed and the respective external phase as well as the fineness of the emulsion. Exemplary inventively usable emulsifiers are
mixtures of alkyl (oligo)glucosides and fatty alcohols, for example, the commercial product Montanov® 68,
partial esters of polyols containing 3-6 carbon atoms with saturated fatty acids containing 8 to 22 carbon atoms,
Sterols. Sterols refer to a group of steroids which carry a hydroxyl group on carbon atom 3 of the steroid skeleton and are isolated from both animal tissue (zoosterols) and vegetal fats (phytosterols). Examples of zoosterols are cholesterol and lanosterol. Examples of suitable phytosterols are ergosterol, stigmasterol and sitosterol. Sterols, the mycosterols, are also isolated from fungi and yeasts.
Phospholipids. These include glucose-phospholipids, obtained, for example, as lecithins or phosphatidyl cholines from, e.g., egg yolk or plant seeds (e.g., soya beans).
fatty acid esters of sugars and sugar alcohols such as sorbitol,
polyglycerins and polyglycerin derivatives such as polyglycerin poly-12-hydroxystearate (commercial product Dehymuls® PGPH), and
linear and branched fatty acids containing 8 to 30 carbon atoms and their Na, K, ammonium, Ca, Mg and Zn salts.

The compositions preferably include emulsifiers in quantities of 0.1 to 25% by weight, particularly 0.5-15% by weight, based on total composition. In a preferred embodiment, polymers are additionally added to the agents, with the addition of cationic polymers having a skin and hair conditioning action being preferred.

Useful cationic polymers refer to polymers that, in their main chain and/or side chain, possess groups that can be “temporarily” or “permanently” cationic. “Permanently cationic” refers to those polymers having a cationic group, independently of the pH of the medium. These are generally polymers which have a quaternary nitrogen atom in the form of an ammonium group, for example. Preferred cationic groups are quaternary ammonium groups. In particular, those polymers wherein the quaternary ammonium groups are bonded through a C₁₋₄ hydrocarbon group to a polymer backbone formed from acrylic acid, methacrylic acid or their derivatives, have proved to be particularly suitable. Homopolymers of the general Formula (VI),

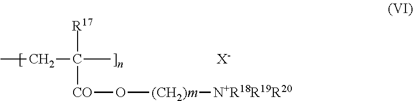

wherein R¹⁷=—H or —CH₃, R¹⁸, R¹⁹ and R²⁰ are, independently of each other, chosen from C₁₋₄ alkyl, -alkenyl or -hydroxyalkyl groups, m=1, 2, 3 or 4, n is a natural number, and X⁻ is a physiologically acceptable organic or inorganic anion, as well as copolymers consisting essentially of the monomer units listed in Formula (III), as well as non-ionic monomer units, are particularly preferred cationic polymers. Regarding these polymers, those that are preferred meet at least one of the following conditions:
R¹⁷ is a methyl group,
R¹⁸, R¹⁹ and R²⁰ are methyl groups,
m is 2, and
X⁻ is halide, sulfate, phosphate, methosulfate as well as organic ions such as lactate, citrate, tartrate and acetate ions. Halide ions are preferred, particularly chloride.

A suitable homopolymer is the optionally crosslinked poly(methacryloyloxyethyl-trimethylammonium chloride) (INCI name Polyquaternium-37). An appropriate suitable commercial product is commercially available under the names Salcare® SC 95 (INCI name: Mineral Oil) and tridecyl-polyoxypropylene polyoxyethylene ether (INCI name: PPG-1-Trideceth-6)) and Salcare® SC 96 (INCI name: Propylene Glycol Dicaprylate/Dicaprate) and tridecyl-polyoxypropylene polyoxyethylene ether (INCI name: PPG-1-Trideceth-6)).

Copolymers with monomer units according to formula (VI) preferably comprise acrylamide, methacrylamide, C₁₋₄ alkyl esters of acrylic acid and C₁₋₄ alkyl esters of methacrylic acid as the non-ionic monomer units. Acrylamide is particularly preferred among these non-ionic monomers. An inventively preferred copolymer is the crosslinked acrylamide-methacryloyloxyethyltrimethylammonium chloride copolymer, such as the copolymer commercially available under the name Salcare® SC 92. Further preferred cationic polymers include
quaternized cellulose derivatives, commercially available under the names Celquat® and Polymer JR®. The compounds Celquat® H 100, Celquat® L 200 and Polymer JR®400 are preferred quaternized cellulose derivatives,
hydrophobically modified cellulose derivatives, for example the cationic polymers marketed under the trade name SoftCat®,
cationic alkyl polyglycosides,
cationised honey, for example the commercial product Honeyquat® 50,
cationic guar derivatives, such as in particular the products marketed under the trade names Cosmedia® Guar and Jaguar®,
polysiloxanes with quaternary groups, such as, for example, the commercially available products Q2-7224 (manufacturer: Dow Corning; a stabilised trimethylsilylamodimethicone), Dow Corning® 929 emulsion (comprising a hydroxylamino-modified silicone, also referred to as amodimethicone), SM-2059 (manufacturer: General Electric), SLM-55067 (manufacturer: Wacker), and Abil®-Quat 3270 and 3272 (manufacturer: Th. Goldschmidt; diquaternary polydimethylsiloxanes, Quaternium-80),
polymeric dimethyldiallylammonium salts and their copolymers with esters and amides of acrylic acid and methacrylic acid such as for example the commercial product Merquat®100 (poly(dimethyldiallylammonium chloride)) and Merquat®550,
copolymers of vinyl pyrrolidone with quaternized derivatives of dialkylaminoalkyl acrylate and dialkylaminoalkyl methacrylate, such as vinyl pyrrolidone-dimethylaminoethyl methacrylate copolymers quaternized with diethyl sulfate. Such compounds are commercially available under the trade names Gafquat® 734 and Gafquat® 755,
vinyl pyrrolidone-vinyl imidazolium methochloride copolymers, as are offered under the trade names Luviquat® FC 370, FC 550, FC 905 and HM 552,
quaternized polyvinyl alcohol,
as well as polymers containing quaternary nitrogen atoms in the main polymer chain, known under the names Polyquaternium 2, Polyquaternium 17, Polyquaternium 18 and Polyquaternium 27.

Polymers designated as Polyquaternium-24 (commercial product e.g. Quatrisoft® LM 200) can also be employed as the cationic polymers. The copolymers of vinyl pyrrolidone are also usable according to the invention, such as the commercially available products Copolymer 845 (manufacturer: ISP), Gaffix® VC 713 (manufacturer: ISP), Gafquat®ASCP 1011, Gafquat®HS 110, Luviquat®8155 and Luviquat® MS 370. In a particularly preferred embodiment of the invention, at least one polymer from the group of the cationic guar derivatives and/or Polyquaternium-7 (Merquat 550), Polyquaternium-6, Polyquaternium-10 and/or Polyquaternium-67 (SoftCat®-Polymers) is comprised as the cationic polymer in the agents according to the invention. Compositions according to the invention preferably contain cationic polymer(s) in quantities of 0.01 to 5 wt. %, based on total composition. Quantities of 0.2 to 3% by weight, particularly 0.5 to 2% by weight, are particularly preferred.

A second subject matter of the present invention is use of a particularly mild surfactant combination of

(a) at least one acylamino acid salt,
(b) at least one alkyl polyglucoside, and
(c) at least one zwitterionic and/or amphoteric surfactant,
in a ratio of components (a):(b):(c) of (0.1-1):(1-3):(0.1-5) in sulfate-free, cosmetic treatment agents for skin and/or hair for improving the compatibility to skin.

A third subject matter of the invention is use of a particularly mild surfactant combination of

(a) at least one acylamino acid salt,
(b) at least one alkyl polyglucoside, and
(c) at least one zwitterionic and/or amphoteric surfactant,
in a ratio of components (a):(b):(c) of (0.1-1):(1-3):(0.1-5) for manufacturing a sulfate-free, cosmetic treatment agent for skin and/or hair which forms a particularly fine pored and creamy foam.

A fourth subject matter of the invention is use of a particularly mild surfactant combination of

(a) at least one acylamino acid salt,
(b) at least one alkyl polyglucoside,
(c) at least one zwitterionic and/or amphoteric surfactant and
(d) at least one compound according to Formula (X),

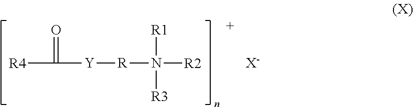

wherein
R4 is a long aliphatic hydrocarbon group containing 8 to 24 carbon atoms,
Y is a hetero atom chosen from O, N or S, wherein when Y═N, the nitrogen atom carries a further substituent —H or —C₁-C₄ alkyl,
R is a straight chain or branched alkylene group containing 1 to 10 carbon atoms,
R1, R2 and R3 are, independently of one another, C₁-C₈ alkyl, C₁-C₈ hydroxyalkyl or benzyl groups, and
X is an anion such as chloride, bromide, methosulfate, ethosulfate, tosylate, acetate, lactate, phosphate or nitrate,
   for improving the feel, combability and suppleness of keratinic fibers.

A fifth subject matter of the invention is a method for cleaning and caring for human skin and human hair wherein an agent according to the invention is applied onto the wet hair and after a contact time of 10 seconds to 5 minutes is rinsed out again.

The following examples illustrate the invention without, however, restricting it in any way. All data are in weight percent, unless otherwise stated.

### EXAMPLES

### 1) Shampoo

| | Quantity |
|---|---|
| Coco-Glucoside | 5.2 |
| Sodium Cocoamphoacetate | 4.7 |
| Disodium Cocoyl Glutamate | 4.0 |
| Citric Acid | 1.6 |
| Sodium Chloride | 1.0 |
| Xanthan Gum | 1.0 |
| Palmitamidopropyltrimonium Chloride | 0.6 |
| Sodium Benzoate | 0.5 |
| Propylene Glycol | 0.4 |
| Salicylic Acid | 0.2 |
| Perfume | 0.2 |
| Aqua | ad 100 |

### 2) Hair Conditioning Shampoo

| | Quantity |
|---|---|
| Coco-Glucoside | 5.2 |
| Cocamidopropyl Betaine | 7.6 |
| Disodium Cocoyl Glutamate | 4.0 |
| Citric Acid | 1.6 |
| Sodium Chloride | 1.0 |
| Xanthan Gum | 1.0 |
| Palmitamidopropyltrimonium Chloride | 0.6 |
| Laurdimonium Hydroxypropyl Hydrolyzed Wheat Protein | 0.5 |
| Sodium Benzoate | 0.5 |
| Propylene Glycol | 0.4 |
| Salicylic Acid | 0.2 |
| Perfume | 0.2 |
| Preservatives | 0.05 |
| Aqua | ad 100 |

We claim:

1. Sulfate-free cleaning agent for skin and/or hair comprising in a cosmetically acceptable carrier a mixture of mild surfactants containing
(a) at least one acylamino acid salt,
(b) at least one alkyl polyglucoside and
(c) at least one zwitterionic and/or amphoteric surfactant,
wherein the ratio of the components (a):(b):(c) is in the range (0.1-1):(1-3):(0.1-5).

2. Cleaning agent for skin and/or hair according to claim 1, wherein the agent is free of compounds of the following Formulae:
H—(O—CH₂CH₂)ₙ—OH (I),
wherein n is a whole number from 1 to 100,000,
H—(O—CH(CH₃)CH₂)ₙ—OH (II),
wherein n is a whole number from 1 to 100,000,
R—(O—CH₂CH₂)ₙ—OH (III),
wherein R is an alkyl or alkenyl group and n is a number from 1 to 10,000,
R—(O—CH₂CH₂)ₙ—OSO₃H (IV),
wherein R is an alkyl or alkenyl group and n is a number from 1 to 10,000, and
—(O—CH₂CH₂)ₙ— (V),
wherein n is a whole number from 2 to 100,000.

3. Cleaning agent for skin and/or hair according to claim 1 wherein the at least one acylamino acid salt (a) is present in an amount of 0.001 to 15 wt. %, the at least one alkyl polyglucoside (b) is present in an amount of 0.05 to 15 wt. %, and the at least one zwitterionic and/or amphoteric surfactant (c) is present in an amount of 0.05 to 20 wt. %, each based on total weight of the agent.

4. Cleaning agent for skin and/or hair according to claim 1 wherein the at least one acylamino acid salt is at least a compound according to Formula (VI), 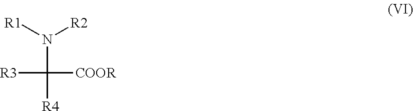
wherein
R is a hydrogen atom, an alkali metal ion or alkaline earth metal ion, an ammonium-, alkylammonium- or alkanolammonium ion,
one of R1 or R2 is hydrogen, a C₁-C₄ alkyl, phenyl or a —CH₂—COOR group, and the other R1 or R2 is a COR′ group, wherein R′ is a straight chain or branched, saturated or unsaturated alkyl group containing 8 to 30 carbon atoms,
R3 and R4 are, independently of one another, hydrogen or linear or branched groups —CH(R5)-(CH₂)ₙ—COOR, wherein R5 is hydrogen or a C₁-C₄ alkyl group and n is a whole number from 0 to 10.

5. Cleaning agent for skin and/or hair according to claim 1 further comprising one or more amphoteric surfactants chosen from N-alkylglycines, N-alkylpropionic acids, N-alkylamino butyric acids, N-alkylimino dipropionic acids, N-hydroxyethyl-N-alkylamido propylglycines, N-alkyltaurines, N-alkylsarcosines, 2-alkylamino propionic acids having about 8 to 24 carbon atoms in the alkyl group, 2-alkylamino acetic acids having about 8 to 24 carbon atoms in the alkyl group, N-cocoalkylamino propionate, cocoacylaminoethylamino propionate, C₁₂-C₁₈ acylsarcosine, N-alkyl-N,N-dimethylammonium glycinates, N-acyl-aminopropyl-N,N-dimethylammonium glycinates, 2-alkyl-3-carboxymethyl-3-hydroxyethyl-imidazolines having 8 to 18 carbon atoms in the alkyl or acyl group, cocoacylaminoethylhydroxyethylcarboxymethyl glycinate, compounds known under the INCI name Cocamidopropyl Betaine, and/or compounds known under the INCI name Disodium Cocoamphodiacetate.

6. Cleaning agent for skin and/or hair according to claim 1 further comprising a compound according to Formula (X), 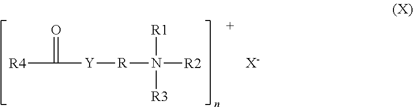
wherein
R4 is a long aliphatic hydrocarbon group containing 8 to 24 carbon atoms,
Y is a hetero atom, selected from O, N or S, wherein when Y═N, the nitrogen atom carries a further substituent —H or —C₁-C₄ alkyl,
R is a straight chain or branched alkylene group containing 1 to 10 carbon atoms,
R1, R2 and R3 are, independently of one another, C₁-C₈ alkyl, C₁-C₈ hydroxyalkyl or benzyl groups, and
X is an anion such as chloride, bromide, methosulfate, ethosulfate, tosylate, acetate, lactate, phosphate or nitrate.

7. Cleaning agent for skin and/or hair according to claim 6, wherein the compound according to Formula (X) is present in an amount of 0.005 to 10 wt. %, based on total weight of the agent, wherein R1, R2 and R3 are identical groups; X is chloride, bromide or methosulfate; R is a straight chain alkylene group —(CH₂)n— in which n=1, 2 or 3; Y is —N(R)—; and R4 is a mixture of different chain lengths from 8 to 24 carbon atoms resulting from the manufacture of compounds of Formula (X) from naturally occurring raw materials.

8. Cleaning agent for skin and/or hair according to claim 1 substantially comprising as a cleaning component only a mixture of surfactants of components (a), (b) and (c).

9. Cleaning agent for skin and/or hair according to claim 1 further comprising at least one consistency regulator.

10. Cleaning agent for skin and/or hair according to claim 1 further comprising at least one vegetal oil component, plant extract and/or protein hydrolyzate.

11. Method for cleaning and caring for human skin and human hair comprising applying an agent according to claim 1 onto wet hair and, after a contact time of 10 seconds to 5 minutes, rinsing the agent out.
